(19) United States
(12) Patent Application Publication
(10) Pub. No.: US 2017/0325829 A1
(43) Pub. Date: Nov. 16, 2017

(54) SNARE

(71) Applicant: THE CATHOLIC UNIVERSITY OF KOREA INDUSTRY-ACADEMIC COOPERATION FOUNDATION, Seoul (KR)

(72) Inventor: Hiun Suk CHAE, Seoul (KR)

(73) Assignee: THE CATHOLIC UNIVERSITY OF KOREA INDUSTRY-ACADEMIC COOPERATION FOUNDATION, Seoul (KR)

(21) Appl. No.: 15/532,830

(22) PCT Filed: Oct. 13, 2015
(86) PCT No.: PCT/KR2015/010797

§ 371 Date: Jun. 2, 2017

(30) Foreign Application Priority Data
Dec. 5, 2014 (KR) 10-2014-0174215

(51) Int. Cl. A61B 17/221; A61B 18/14; A61B 17/3205; A61B 18/00

(52) U.S. Cl. CPC A61B 17/221; A61B 18/1492; A61B 17/3205; A61B 2018/00577; A61B 2017/2212

(57) ABSTRACT

Disclosed herein is a snare. The snare includes: a tube injected into a body; a wire movably inserted into the tube; a noose joined to a leading end of the wire and ablating a polyp as an internal area expands or contracts by passing through an end of the tube; and at least one identification marker formed along the noose.

### TECHNICAL FIELD

The present disclosure relates to a snare, and more particularly, to a snare having an identification marker provided in a noose thereof to determine a size of the noose, thereby adjusting the size of the noose according to a size of polyps and more easily ablating the polyps along an ablation direction of the polyps.

### BACKGROUND ART

In general, an endoscope is a medical device that is inserted into a body of a subject to check or examine affected areas with the naked eye, and a snare is used to ablate protruding tissues such as polyps found by the endoscope.

The snare is inserted into the body of the subject using the endoscope inserted into the body of the subject as a guide means.

Meanwhile, referring to FIG. 1, the snare includes a wire 3 inserted into a tube 2 of a flexible insulating material and an operating tool 4 connected to one end of the wire 3. The other end of the wire 3 is provided with a noose 1 and the operating tool 4 is operated to push and pull the wire 3 so that the noose 1 is drawn into and out from the end of the tube 2.

As the noose 1 is drawn out from the end of the tube 3, a size of an internal area of the noose 1 is getting larger and if the nose 1 is drawn inward from the end of the tube 3, the size of the internal area of the noose 1 is reduced due to an aperture of the tube 3.

That is, the snare draws out the noose 1 toward an outside of the end of the tube 3 to increase the size of the internal area of the noose 1 and then make protruding tissues such as the polyps be inserted into the internal area of the noose 1, and operates the operating tool 4 to pull the wire 3 to reduce the internal area of the noose 1, thereby ablating the protruding tissues such as the polyps.

Meanwhile, when the noose 1 of the snare is drawn out toward the outside of the end of the tube 2 to ablate polyps, the size of the noose may be expanded unlike the size of the polyps. If the size of the noose 1 is much larger than that of the polyps, a serous membrane is caught together with the polyps while the size of the noose 1 is decreasing to ablate the polyps, such that the ablation of the polyps is not performed, whereas if the size of the noose 1 is expanded smaller than the size of the polyps, only a part of the polyps is ablated, such that the ablation of the polyps is not performed properly.

Therefore, it is necessary to adjust the size of the noose to fit the size of the polyps when the size of the noose is increased to insert the polyps into the internal area of the noose. In the case of the existing snare, the size of the noose may not be guessed during the polyp ablation operation, and therefore there is a problem in that the ablation of the polyps is not performed properly during the operation.

### DISCLOSURE

### Technical Problem

The present disclosure relatives to a snare having an identification marker provided in a noose thereof to determine a size of the noose, thereby adjusting the size of the noose to fit a size of polyps and more easily ablating the polyps along an ablation direction of the polyps.

### Technical Solution

According to an exemplary embodiment of the present disclosure, a snare includes: a tube injected into a body; a wire movably inserted into the tube; a noose joined to a leading end of the wire and ablating a polyp as an internal area expands or contracts by passing through an end of the tube; and at least one identification marker formed along the noose.

The identification marker may be arranged so that being exposed to an outside of the end of the tube according to a change in a size of the internal area of the noose.

An inner surface of the noose may be provided with at least one ablation protrusion for ablating the polyp.

An outer surface of the ablation protrusion may be provided with a teeth portion.

An inner surface of the noose may be provided with at least one support protrusion for fixing the noose to the polyp.

A surface of the support protrusion facing the polyp may be formed to be rounded.

The support protrusion may be formed in plural and the plurality of support protrusions may be placed in pair to face each other at one end and the other end of the noose.

An ablation protrusion for ablating the polyp may be formed between the support protrusions.

The leading end of the wire may be provided with a fixing needle that is toward the internal area of the noose and fixes the noose.

### Advantageous Effects

According to the exemplary embodiments of the present disclosure, it is possible to provide the identification marker in the noose of the snare to determine the size of the noose, thereby adjusting the size of the noose to fit the size of the polyps and more easily ablating the polyps along the ablation direction of the polyps.

### DESCRIPTION OF DRAWINGS

FIG. 1 is a schematic view of a snare according to the related art.

FIGS. 2 and 3 are diagrams showing a use state of a snare according to an exemplary embodiment of the present disclosure.

FIGS. 4 to 6 are diagrams schematically showing a snare according to a modification of the exemplary embodiment of the present disclosure.

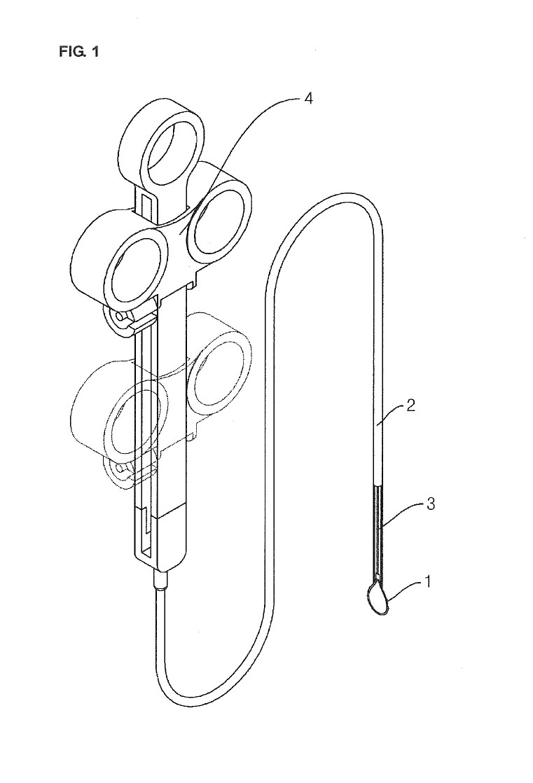

### BEST MODE

Since the present disclosure may be variously modified and have several exemplary embodiments, specific exemplary embodiments will be illustrated in the accompanying drawings and be described in detail in a detailed description. However, it is to be understood that the present disclosure is not limited to the specific exemplary embodiments, but includes all modifications, equivalents, and substitutions included in the spirit and the scope of the present disclosure. Further, when it is determined that the detailed description of the known art related to the present disclosure may obscure the gist of the present disclosure, the detailed description thereof will be omitted.

Hereinafter, a snare according to an exemplary embodiment of the present disclosure will be described in detail with reference to the accompanying drawings. In describing a snare according to an exemplary embodiment of the present disclosure with reference to the accompanying drawings, components that are the same as or correspond to each other will be denoted by the same reference numerals, and an overlapped description thereof will be omitted.

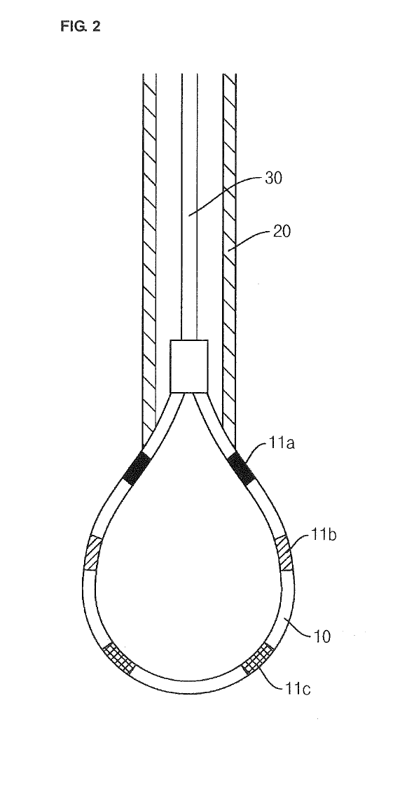

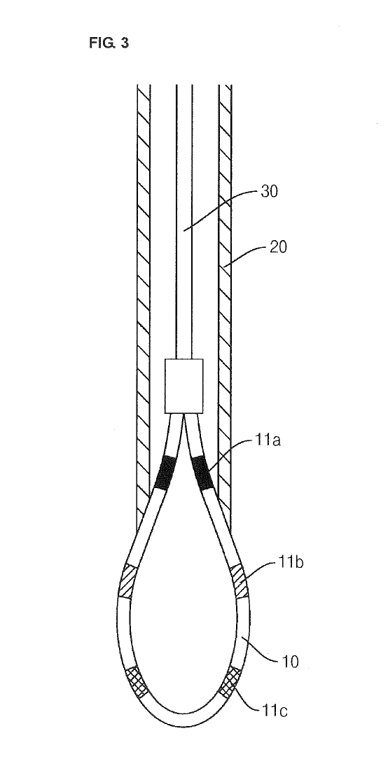

FIGS. 2 and 3 are diagrams showing a use state of a snare according to an exemplary embodiment of the present disclosure.

FIGS. 2 and 3 illustrate a tube 20, a wire 30, a noose 10, and identification markers 11a, 11b and 11c.

The tube 20 is injected into a body. The tube 20 of a flexible material is elongated in a forward/backward direction and inserted into the body together with an endoscope (not illustrated).

If positions of polyps are confirmed by an endoscope (not illustrated), the polyps are ablated using the noose 10 to be described later.

The wire 30 is movably inserted into the tube 20. The wire 30 is made of a metallic material having elasticity, and the wire 30 inserted back and forth is inserted into the body together with the tube 20 to move, and if the polyps are found by the endoscope (not illustrated) the wire 30 is moved forwardly by an operating tool 4 and the noose 10 joined to the wire is expanded while being drawn out to the outside of the tube 20 and is contracted while being drawn in the inside of the tube 20.

The noose 10 is joined to a leading end of the wire 30 and ablates polyps as an internal area expands and contracts by passing through an end of the tube 20. The noose 10 has a substantially elliptical ring shape, and the term “internal area” means a space having a varying size as a space inside the ring-shaped noose 10.

The noose 10 is drawn in and out from the end of the tube 20 according to the movement of the wire 30 moving by the operating tool 4. The noose 10 is exposed to the outside when it is drawn out from the end of the tube 20 so that the size of the noose 10 is gradually increased while the internal area of the noose 10 is formed in an elliptical shape and the noose 10 is elastically deformed by the tube 20 when it is drawn in the tube 20 so that the size of the internal area is gradually decreased to insert the noose 10 into the tube 20.

Specifically, if positions of polyps are confirmed by the endoscope, the operating tool 4 is operated by an operator to move the wire 30 forward and expose the noose 10 to the outside of the tube 20 while the wire 30 moves forwardly, such that the internal area of the noose 10 has an elliptical shape. The exposed noose 10 is moved so that polyps are positioned in the internal area of the noose 10. Thereafter, the operating tool 4 is operated by an operator to move the wire 30 backward and draw the noose 10 in the tube 20 while the wire 30 moves backward. The size of the internal area of the noose 10 is decreased as the noose 10 is drawn in the tube 20, such that the noose 10 ablates the polyps.

At least one identification marker 11a, 11b, or 11c is formed along the noose 10. The identification markers 11a, 11b and 11c are markers formed on the noose 10 so that the noose 10 is identified in the body and identify blood due to bleeding during the operation, ablated polyps, and the noose 10.

Meanwhile, the identification markers 11a, 11b, and 11c may be arranged so that different colors are exposed to the outside of the end of the tube 20 according to the change in the size of the internal area of the noose 10. At this time, the identification markers 11a, 11b, and 11c may have different fluorescent colors and may be arranged in pairs in the noose 10.

Referring to FIGS. 2 and 3, the size of the internal area of the noose 10 may be determined according to the colors of the identification markers 11a, 11b, and 11c.

FIG. 2 illustrates the case where the identification marker 11a is exposed to the outside of the end of the tube 20 and the size of the noose 10 is the largest and FIG. 3 illustrates that the identification marker 11b is exposed to the outside of the end of the tube 20 and the size of the noose 10 is middle. Although not illustrated, when only the identification marker 11c is exposed to the outside of the end of the tube 20, the size of the noose 10 is the smallest.

In this way, the size of the noose 10 may be determined by looking at the identification markers 11a, 11b, and 11c exposed to the outside of the end of the tube 20.

At this time, if the size of the internal area of the noose 10 is understood in advance and the colors of the identification markers 11a, 11b, and 11c are arranged, the size of the noose 10 may be understood more accurately. For example, if the first identification marker 11a is placed at a position where a length of the internal area of the noose 10 is 3 cm, the second identification marker 11b is placed at a position where the length of the internal area of the noose 10 is 2 cm, and the third identification marker 11c is placed at a position where the length of the internal area of the noose 10 is 1 cm, it may be determined that the noose 10 has been drawn 3 cm, 2 cm, or 1 cm from the end of the tube 20 according to the colors of the identification markers 11a, 11b, and 11c that are exposed.

Meanwhile, when the noose 10 of the snare is drawn out toward the outside of the end of the tube 20 to ablate the polyps, the size of the noose may be larger than the size of the polyps. If the size of the noose 10 is much larger than that of the polyps, a serous membrane is caught together with the polyps while the noose 10 is narrowing to ablate the polyps, such that the ablation of the polyps is not performed, whereas if the size of the noose 10 is slightly larger than that of the polyps, only a part of the polyps is ablated, such that the ablation of the polyps is not performed properly.

The snare according to the exemplary embodiment of the present disclosure uses the identification markers 11a, 11b, and 11c to adjust the size of the noose 10 according to the size of the polyps captured by the endoscope, such that the noose 10 may accurately ablate only the polyps. In addition, the polyps to be ablated and the noose 10 are identified from each other during the operation by using the identification markers 11a, 11b, and 11c, thereby facilitating the polyp ablation operation.

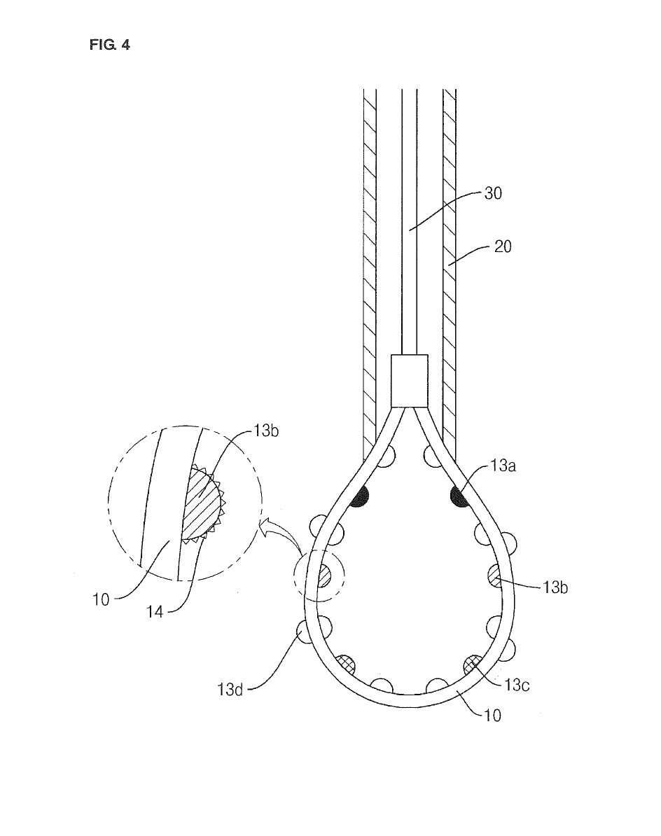

Meanwhile, referring to FIG. 4, the snare according to the exemplary embodiment of the present disclosure may include at least one ablation protrusion 13a, 13b, or 13c for ablating the polyps on the inner surface of the noose 10.

The ablation protrusions 13a, 13b and 13c are formed on the inner surface of the noose 10 so that polyps positioned in the internal area of the noose 10 may be easily ablated.

Although the exemplary embodiment of the present disclosure has been described with reference to the case where the ablation protrusions 13a, 13b and 13c are formed to protrude on the inner surface of the noose 10, it is also possible that the ablation protrusion 13d is formed to protrude on the outer surface of the noose 10.

In FIG. 4, the ablation protrusions 13a, 13b, and 13c have a semicircular shape, but may have various shapes such as a rectangle as long as they protrude from the inner surface or the outer surface of the noose 10 to ablate the polyps.

In addition, the outer surfaces of the ablation protrusions 13a, 13b, 13c, and 13d may be provided with a teeth portion 14. The plurality of teeth portions 14 may protrude from the outer surfaces of the ablation protrusions 13a, 13b, 13c, and 13d so that the polyps may be easily ablated.

Meanwhile, the identification markers 11a, 11b and 11c having different colors are formed on the outer surfaces of the ablation protrusions 13a, 13b, 13c and 13d according to the change in the size of the internal area of the noose 10, such that the size of the noose 10 may be understood accurately during the operation.

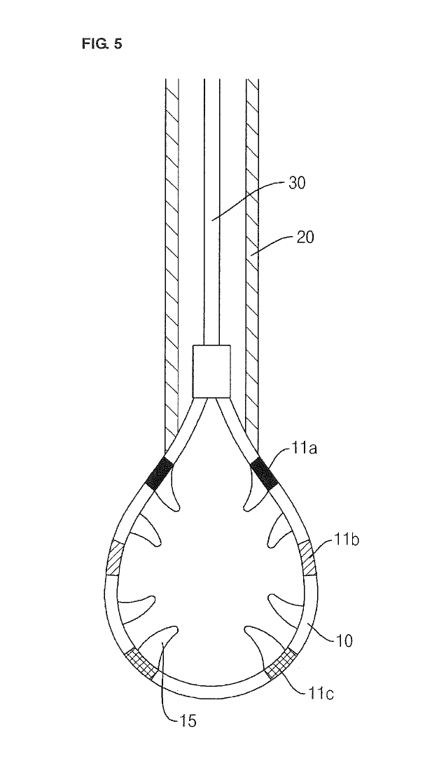

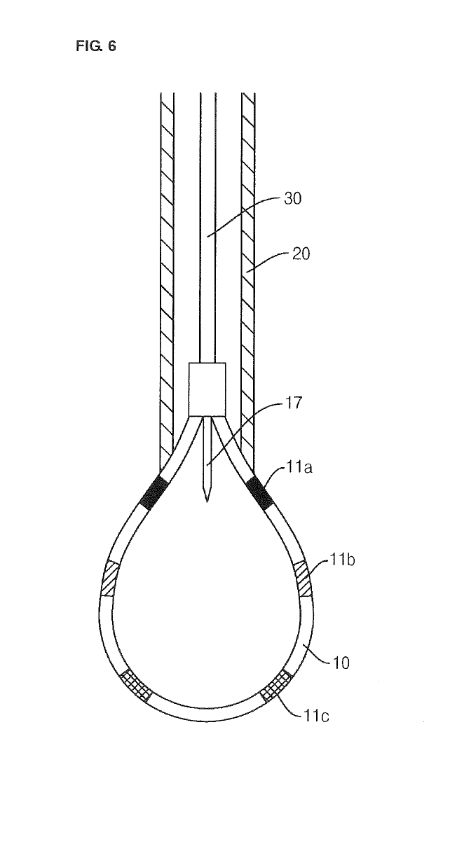

Meanwhile, referring to FIG. 5, in the snare according to the exemplary embodiment of the present disclosure, the inner surface of the noose 10 may be provided with at least one support protrusion 15 for fixing the noose 10 to the polyp.

The support protrusion 15 is formed to protrude on the inner surface of the noose 10, and the end of the support protrusion 15 supports the polyp when the polyp is inserted into the internal area of the noose 10 in order to ablate the polyp, thereby fixing the noose 10 to the polyp. A surface of the support protrusion 15 that faces the polyp may be formed to be rounded. The support protrusion 15 may have a hook shape, and the surface of the support protrusion 15 that faces the polyp protrudes and is formed to be rounded and the direction thereof may be the same as or opposite to the ablation direction. The polyp may be fixed to the noose 10 by the rounded surface. At this time, the support protrusion 15 may be formed in plural, and may be arranged in pairs so as to face each other at one end and the other end of the snare 10.

Further, the noose 10 may be provided with the ablation protrusions 13a, 13b and 13c for ablating the polyps between the support protrusions 15 and the ablation protrusions 13a, 13b, and 13c are used in the state where the noose 10 is fixed to the polyps by the support protrusions 15 to ablate the polyps, such that the snare may easily ablate the polyps.

Meanwhile, the leading end of the wire 30 may be provided with a fixing needle 17 that is toward the internal area of the noose 10 and fixes the noose 10 to the polyps.

The fixing needle 17 is formed to protrude in the direction of the internal area of the noose 10 from the leading end of the wire 30 by a predetermined length, and if the polyp is placed in the internal area of the noose 10 to be ablated and then the wire 30 moves forwardly, the polyps are fixed at a proper position when the fixing needle 17 pricks the polyps and thus the noose 10 ablates the polyps. Thereafter, if the wire 30 moves backward, the internal area of the noose 10 is gradually decreased to ablate the polyps.

Although the preferred exemplary embodiments of the present disclosure have been disclosed for illustrative purposes, those skilled in the art will appreciate that various modifications, additions and substitutions are possible, without departing from the scope and spirit of the invention as disclosed in the accompanying claims. Accordingly, such modifications, additions and substitutions should easily be understood to fall within the scope of the present disclosure.

1. A snare, comprising:
a tube injected into a body;
a wire movably inserted into the tube;
a noose joined to a leading end of the wire and ablating a polyp as an internal area expands or contracts by passing through an end of the tube; and
at least one identification marker formed along the noose.

2. The snare of claim 1, wherein the identification marker is arranged so that different colors are exposed to an outside of the end of the tube according to a change in a size of the internal area of the noose.

3. The snare of claim 1, wherein an inner surface of the noose is provided with at least one ablation protrusion for ablating the polyp.

4. The snare of claim 3, wherein an outer surface of the ablation protrusion is provided with a teeth portion.

5. The snare of claim 1, wherein an inner surface of the noose is provided with at least one support protrusion for fixing the noose to the polyp.

6. The snare of claim 5, wherein a surface of the support protrusion facing the polyp is formed to be rounded.

7. The snare of claim 5, wherein the support protrusion is formed in plural and the plurality of support protrusions are placed in pair to face each other at one end and the other end of the noose.

8. The snare of claim 7, wherein an ablation protrusion for ablating the polyp is formed between the support protrusions.

9. The snare of claim 1, wherein the leading end of the wire is provided with a fixing needle that is toward the internal area of the noose and fixes the noose.
